(19) United States
(12) Patent Application Publication
(10) Pub. No.: US 2013/0345113 A1
(43) Pub. Date: Dec. 26, 2013

(54) Method of Use of Activated Functional Proteins to Improve Animal Health

(71) Applicant: Ronald E. Strohbehn, Nevada, IA (US)

(72) Inventor: Ronald E. Strohbehn, Nevada, IA (US)

(21) Appl. No.: 13/182,018

(22) Filed: Jul. 13, 2011

(51) Int. Cl. A61K 38/00

(52) U.S. Cl. 514/1.1

(57) ABSTRACT

It was found that feeding a composition including activated growth factor(s) increases feed to gain ratio, increases overall weight gain, reduces necessary antibiotic or electrolyte therapy and reduces mortality in animals. The composition is derived by first separating growth factor(s) from a source such as whey or blood, then subjecting the factor to an activation process, and then providing the activated growth factor to the animal. A feed additive comprising activated growth factors in appropriate amounts, shows results that are an improvement over standard therapies of supplementation. Application of activated growth factors may be by topical, injection or oral application.

### FIELD OF INVENTION

The present invention is related to the application and use of activated functional proteins to promote growth of the animals. More specifically, the invention is related to animal feed compositions and supplements that include activated functional proteins and discloses a method for feeding the same to promote growth, health, weight gain, feed efficiency, feed intake, and improved feed to gain ratios as well as reduce the use of antibiotics and other medicinal preparations.

### BACKGROUND

A “functional protein” is a protein that provides more than nutritional or caloric value. Functional proteins may regulate, control, or otherwise affect metabolic or immune status. Examples of functional proteins as used hereinafter are lysozymes, lactoferrin, growth factors, transfer factors, cytokines, and immuoglobulins. This listing is not exhaustive, but for the purpose of example. Growth factors and cytokines are involved in cell signaling, differentiation and growth. It is thought that cytokines effect the levels and functionality of interleukins. Communication at the cellular level involves cytokines binding to specific receptors on the cell surface. Growth factors and cytokines regulate a variety of cellular and metabolic processes including those related to interleukins. They mediate embryonic development, tissue growth, tissue repair and wound healing.

Growth factors are part of a complex family of peptide hormones or biological compounds such as transforming growth factors, insulin-like growth factors, myostatin, epithelial growth factor, and placental growth factor specifically such as IGF-1. The use of growth factors in therapeutic settings for treatment of a number of human conditions has increased over the last decade, and this trend is expected to continue. Furthermore, utilization of these compounds in animal feed, injection, and topical applications seem likely as well.

Insulin-like growth factors (IGFs) are polypeptides with high sequence similarity to insulin. IGF's are part of a complex system that are important for both regulation of normal physiology, as well as a number of pathological states, including cancer, and play a role in cell proliferation and inhibition of cell death. IGF may affect different growth stages. Insulin-like growth factor 2 (IGF-2) is thought to be a primary growth factor required for early development while insulin-like growth factor 1 (IGF-1) expression is required for achieving maximal growth. Almost every cell in the human body is affected by IGF-1, especially cells in muscle, cartilage, bone, liver, kidney, nerves, skin, and lungs. IGF-1 can also regulate cell growth and development, especially in nerve cells, as well as DNA synthesis.

There are a number of methods for separating functional proteins such as growth factors and cytokines from biological materials, and some methods have disclosed ways to obtain activated growth factors and functional proteins. A generally well-known process for separating growth factors from whole blood comprises adding an anti-coagulant to blood, centrifuging, and recovering the plasma. Next, lipids may be extracted from the plasma by a method known to those skilled in the art by chemical precipitation. Growth factors may be further extracted from lipids via pH treatment steps and fractionation.

Alternatively, EP 0313.515 describes a process to purify growth factors in milk. This disclosure employs successive chromatographic steps, specifically cation exchange chromatography. Another process to isolate growth factors from milk includes cation exchange chromatography, followed by hydroxyapatite chromatography, then employing different elutants to obtain various TGF-beta fractions. (see PCT App. WO 0125.276).

In U.S. Pat. No. 7,141,262 growth factors are obtained by diluting milk or whey to a concentration between 5 and 30 g/liter of solution, then precipitating part of the whey proteins at temperatures between about 60C.° and 68C.° with acid treatment to pH between 4 and 5.5. The solution is then diafiltered by microfiltration (MF) resulting in an MF retentate that is highly enriched in TGF-beta. Reference to U.S. Pat. Nos. 7,057,016 and 6,057,430 also provide background disclosures related to processes for obtaining activated growth factors.

It is hypothesized that feeding activated functional proteins to animals or providing them through supplements or topical applications might encourage cell proliferation and growth.

Cereal grains like corn, wheat and rice are commonly fed to livestock and other animals as a composition. Many feed compositions have been developed and utilized to positively impact the health and growth of the animal. For example, a chicken feed composition including pecan byproduct promotes the health and rejuvenates egg production if administered via a specified method (U.S. Pat. No. 7,470,439). A method for increasing breast meat yields in poultry is accomplished by feeding a composition that includes protein, vitamins, minerals, a source of galactoside, and an alpha-galactosidase. This method claims to increase the gain to feed ratio in addition to the amount of white meat relative to feeding protocols that include the feed composition absent the alpha-galactosidase. (U.S. Pat. No. 6,174,558).

A more recent patent issued in September 2010 claims a composition for promoting animal growth. The composition comprises extracts of Artemisia capillaris thunberg, Acanthopanax and garlic. This patent discloses a method of making the extracts. The inventor claims that feeding this composition up-regulates secretion of growth hormone which results in increased body weight gain, meat quality improvement and milk production in livestock animals. The extracts of Artemisia capillaries thunberg, Acanthopanax and garlic should be added to a conventional feed premix consisting of corn, soybean meal, wheat, tallow, molasses, calcium phosphate, limestone, salt, etc, preferably in an amount of 0.005-50% by weight based on the total weight of feed. Extracts were measured in-vitro in a cell culture assay to test the effect of promoting secretion of growth hormone. (U.S. Pat. No. 7,790,206).

It is well understood that the addition of blood plasma to neonatal and starter animal feed can result in some improvement in average daily gain, feed intake and gain to feed ratio. Furthermore, it is known that plasma contains functional proteins including growth factors. However, it is desirable to increase weight gain over and above that resulting from plasma addition to feeds along with feed efficiency and growth. It is also desirable to reduce mortality and improve health of the animal. To achieve these results without upsetting the nutritional balance of regular feed would be preferred. Finally, it would be advantageous to achieve these results with a substance of comparatively less mass for ease of mixing, handling and storage

The first objective of the present invention is to provide a feed additive or supplement that will improve the feed to gain ratio for young animals, increase feed efficiency, positively effect growth and, preferably, increase the overall gain when compared to feeding the standard plasma additive;

The second objective of the present invention is to provide a feed additive, supplement or medicinal application that can be added at a volume or weight which is easy to handle and mix, and still encourages the animal to ingest and maintain the balance of the underlying nutrients in the feed base without displacing them;

The third objective of the present invention is to provide a feed additive, supplement or medicinal application that will improve the overall health of the animal that ingests it as measured by mortality rates and by treatment with antibiotics and electrolytes.

### BRIEF SUMMARY OF THE INVENTION

The general understanding of functional proteins and growth factors is that they affect certain cellular pathways. Some of these pathways control cellular proliferation (growth). It is also generally understood that functional proteins trigger these pathways only when in an activated state. The present invention utilizes activated functional proteins to effect a number of growth and health metrics including, but not limited to, increase gain, feed intake, health, improve feed to gain ratio's, and/or lessen mortality rates. The functional proteins are derived from blood plasma, mammal tissue, animal tissue or milk products but can also be derived from many other biological sources. A biological source is usable in the present invention as long as the starting material contains a functional protein in a latent form, whether bound to a carrier protein or not, and can be activated either by release from its carrier protein or otherwise stimulated.

In the present method, correct use of activated functional proteins may do any or all of the following: reduce mortality rates, increase weight gain, increase feed intake, improve overall health of the animal, and improve feed to gain ratio's.

### DETAILED DESCRIPTION OF THE PRESENT INVENTION

Functional proteins employed in the present invention can be obtained from a number of sources. Specifically, although not intended to be limiting, the following list provides a variety of starting materials or biological sources for consideration, said sources including blood, milk, and tissue from mammals, some specific examples of which are listed here: plasma, milk products, whole blood, red blood cells, mucosa tissue, intestinal tissue, Spray Dried Bovine Plasma (SDBP), Spray Dried Porcine Plasma (SDPP), liquid bovine plasma, liquid porcine plasma, liquid milk, powdered milk, liquid colostrum, spray dried colostrum, liquid whey, whey protein concentrate, milk powder, whey protein isolate, whey retentate, tissue, blood, embryonic tissue. In summary, blood, plasma milk, colostrum, whey products, milk products, and embryonic tissue from all mammalian species; and plasma products from other agricultural animals such as chickens, and other biological tissues from which the functional protein can be separated may be biological sources of functional proteins.

The animals that may be affected by feeding, supplementing or medicinal use of the activated functional protein include bovine, porcine, human, equine, canine, feline, aquacultured fish and crustacae, and poultry.

The functional proteins derived from the biological source may be activated by one of several different methods known in the art which include, but are not limited to methods that employ pH adjustment, heat shock, temperature adjustment, alcohol extraction, enzyme addition, ionic changes, other chemical additions, and pressure, or combinations thereof as disclosed in “Physicochemicl Activation of Recombinant Latent Transforming Growth Factor—beta's 1, 2, and 3”, Brown, Peter D., Wakefield, Lalage M., Leninson, Arthur D., Sporn, Micheal B. (1990) Growth Factors, Vol. 3, pp. 35-43. Several other methods are disclosed in the patent references made herein. Quantitative analyses procedures such as ELISA assays employ methods to activate and measure functional proteins. Further, another method for measuring activated growth protein is disclosed in U.S. Pat. No. 7,094,550. Desirable results were obtained with activated functional proteins wherein the level of activated growth factors was elevated above the natural state of the biological source.

Once the functional proteins are activated, it is desirable to stabilize them in that activated state for addition to the feed or other material for delivery to the animal so that the process can be employed to produce feed or other products which can be stored. However, stabilization is for convenience and is not necessary for producing the desired results and effects as described herein.

In one embodiment, the activated functional proteins are combined with a standard feed composition base where the base is tailored to the species and growth stage of the animal. However, combining with a standard feed composition is not the only mode of delivery. Other modes include topical applications and medicinal preparations for oral use or for injection. As a more specific example, when the activated functional proteins comprise activated growth factors they can be fed at a rate that is, generally, less than that of the recommended rate for feeding most commercially available plasma additives. In this application, the nutritional proteins otherwise provided by a standard plasma additive are replaced. Generally, these nutritional protein replacements are via plant or animal proteins such as soy protein and red blood cells.

In several trials, it was determined that implementing activated functional proteins as a replacement for the standard plasma supplements resulted in a statistically improved feed to gain ratio, higher absolute weight gain, and reduced mortality rates.

In a second embodiment, activated functional proteins were fed as a single-dose neonatal supplement. When compared to animals fed either a colostrum supplement or a standard milk replacer rather than the single-dose neonatal supplement of activated functional proteins, results showed significant decreases in mortality and in required treatment with antibiotics and electrolytes.

Other embodiments include administering the activated functional proteins to increase rate of growth in order to reach full recovery from a growth-inhibiting condition, support growth in normal growth stages, or to assist in recovery from stress or other conditions.

Other objects, features, and advantages of the present invention will be readily appreciated from the following description. The description makes reference to the accompanying drawings, which are provided for illustration of the preferred embodiment. However, such embodiment does not represent the full scope of the invention. The subject matter which the inventor does regard as his invention is particularly pointed out and distinctly claimed in the claims at the conclusion of this specification.

### BRIEF DESCRIPTION OF THE DRAWINGS

FIG. 1 Swine trials Phase 1 diet

FIG. 2 Swine trials Phase 2 diet

FIG. 3 Days 0-21 chart comparing daily gain, daily feed intake, feed to gain ratios

FIG. 4 Days 0-43 chart comparing daily gain, daily feed intake, feed to gain ratios

FIG. 5 Chart neonatal bovine trials

### DETAILED DESCRIPTION OF THE INVENTION

Functional proteins including growth factors were present in plasma purchased commercially, and activated (for an example of an activation process, see Example 3 below), and thereafter dried. The activated growth factors were administered as described in several trials and results were collected. In the Examples below, the terms 1/x diet or 1/x functional proteins or growth factors diet refers to a diet wherein functional proteins and growth factors are fed at a rate that is 1/x that fed of plasma in the plasma diet. In other words, if the plasma diet includes X amount of plasma, then the 1/x diet includes an amount of functional proteins including growth factors that is 1/x the amount of plasma in the plasma diet. The difference in mass and protein was made up with nutirtional protein supplements, often soy derived.

### Example 1—Titration Trial

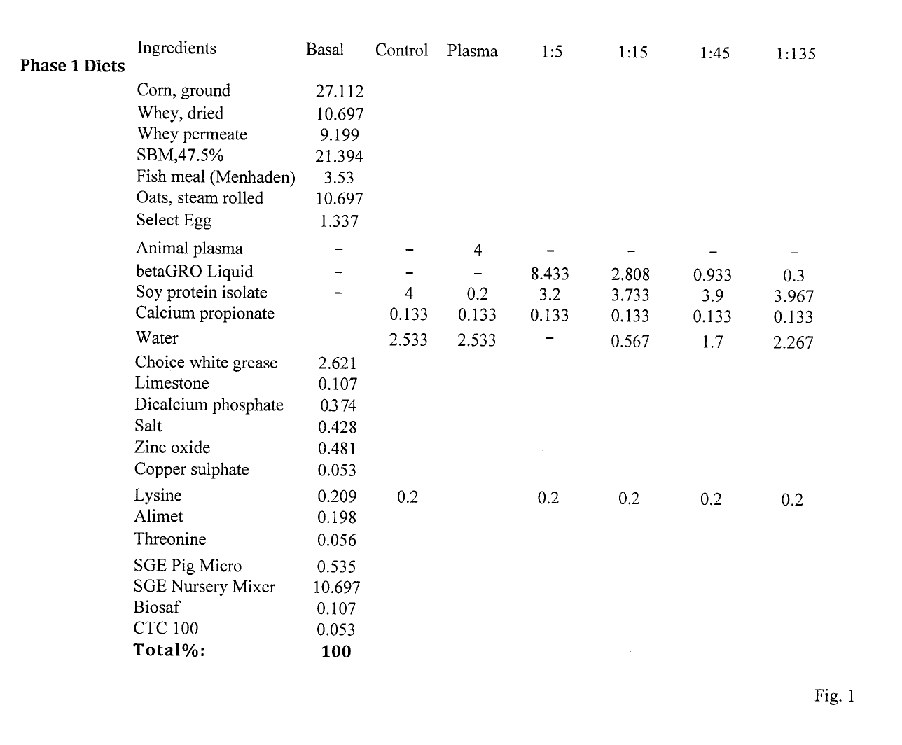

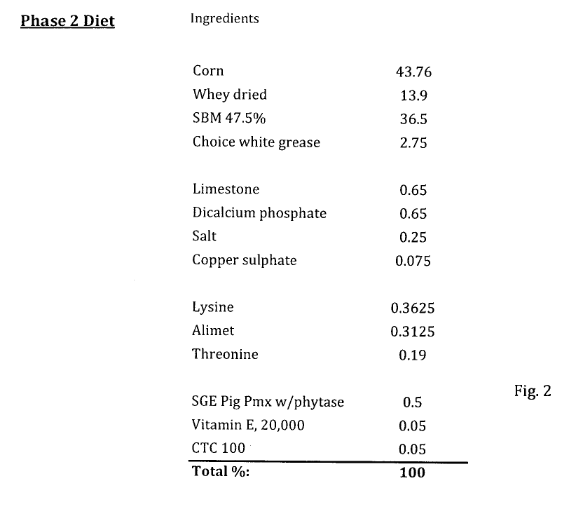

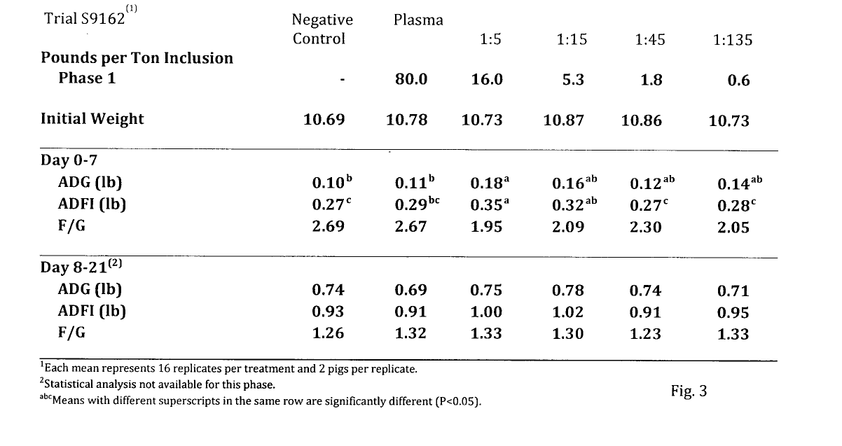

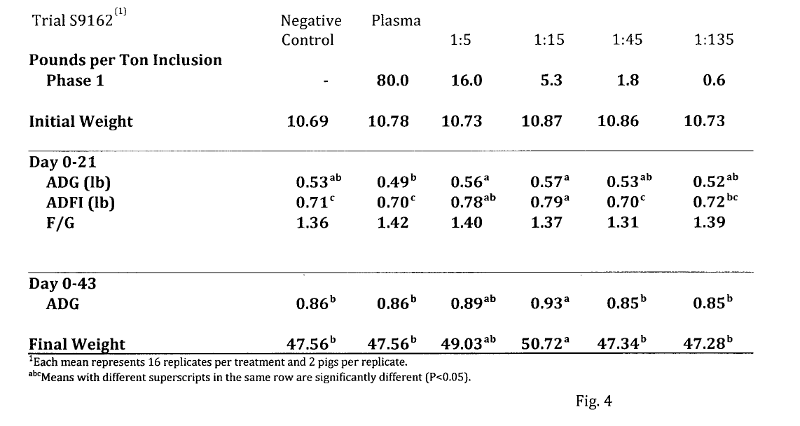

Titration trials were conducted. Referring now to FIGS. 1 and 2, a study of 192 pigs, with an average start weight of 10.8 lbs. was conducted. The study was set for 2 pigs/pen, 16 replicates per treatment. The activated functional proteins diets were fed for 14 days as was a control diet, and a plasma diet. Thereafter all pigs were fed the same diet (see FIGS. 1 and 2). The functional proteins diets were set to include activated functional proteins at specified weight ratios relative to plasma in the plasma diet. The functional proteins diets did not include plasma; however, the protein equivalent of the plasma was added in the form of soy protein isolate. The control diet did not include plasma or activated functional proteins. The results shown in FIG. 3 indicate that the average daily gain for days 0-7 was statistically higher (p<0.05) for the activated functional proteins diet at 1/5 the weight of plasma in the plasma diet. The average daily feed intake in the 1/5 and 1/15 functional proteins diets were statistically higher than for the control diet, but feed intake only for the 1/5 diet was statistically higher than the plasma diet. As a result of these differences, the feed to gain ratios for all of the functional proteins diets were lower than for either the control or the plasma diet for days 0-7, specifically the 1/5 diet. For days 8-21, average daily gain and average daily feed intake were only slightly higher for the activated functional proteins diets, with the 1/5 and 1/15 diets highest. Feed to gain ratios followed suit. Results, overall, for days 1-21 and, again, for days 1-43 are shown in FIG. 4. Average daily gain for the 1/5 and 1/15 functional proteins diet was statistically higher than the plasma diet for days 0-21; average daily gain for the 1/15 functional proteins diet was higher for days 0-43.

### Example 2(5)—Calves Mortality and Treatment

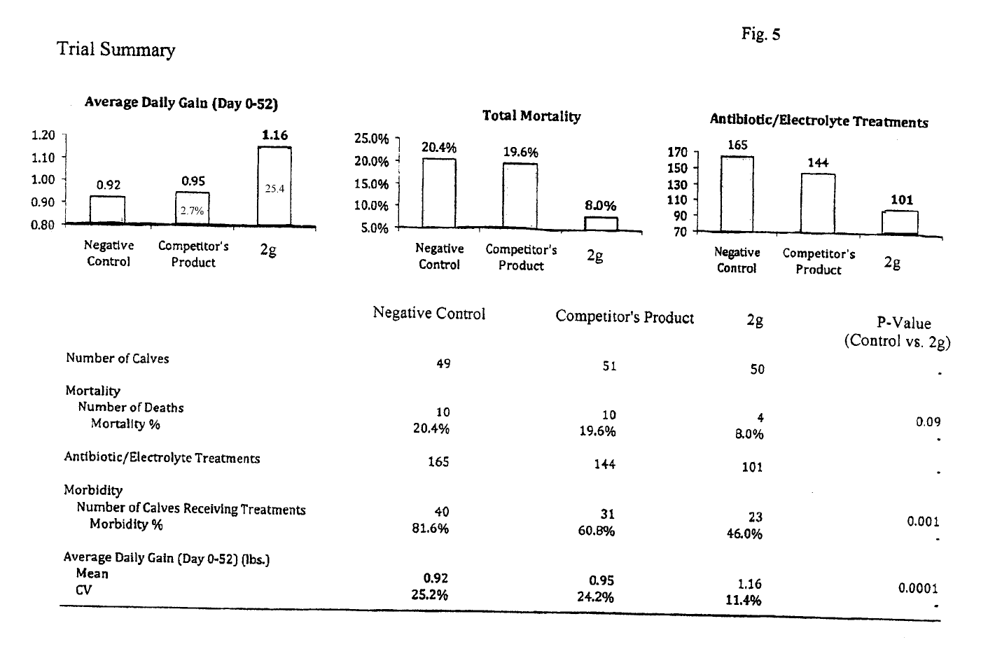

In this study, 150 calves between 0-3 days old were randomly allotted in a single blind study. There were three groups; negative control, competitor colostrum supplement, and the functional proteins blend. The study tracked weight gain, mortality and quantity of antibiotic/electrolyte treatments. The colostrum supplement and the functional proteins blend (2 grams blended with standard milk replacer to normal dose weight) replaced the first standard milk replacer bottle feeding for those respective experimental groups; thereafter, all calves were fed the same standard milk replacer. Weight gain for the activated functional proteins group was higher than the other groups while mortality was less than half that of the colostrum supplement or the negative control. The number of antibiotic and electrolyte treatments required for the activated functional proteins group was only 70% the number required for the colostrum group, and only 61% of the number required by the negative control. (see FIG. 5)

### Example 3—Method to Obtain Activated Growth Factors

Activated growth factors may be obtained as known in the prior art via pressure, pH activation, enzyme addition, ionic changes, other chemical treatment or heat shock following standard protocols. See “Physicochemicl Activation of Recombinant Latent Transforming Growth Factor—beta's 1,2, and 3”, Brown, Peter D., Wakefield, Lalage M., Leninson, Arthur D., Sporn, Micheal B. (1990) Growth Factors, Vol. 3, pp. 35-43.

In the present invention, functional proteins were not separated from the biological starting material employing one of many methods known in the art but were, instead, purchased from a commercial supplier.

The growth factors were then activated by two of the aforementioned methods as described by Brown et al. In one application, pH was adjusted from the starting material's natural pH until measurements of activation of functional proteins showed an increase in activation above the activation at the natural pH. As known in the prior art, the pH adjustment used may be either basic or acidic with sharp transitions from latency between pH 4.1 and 3.1 and between pH 11.0 and 11.9 as reported by Brown et al. for both TGF-β1 and β2. As is also well known, adjustments can be achieved via standard addition of NaOH, HCl or other bases and acids.

The second activation method used included heat shocking the commercially obained proteins to 75 degrees Celsius, holding for five (5) minutes or to 80 degrees Celsius for 1 minute, or until measurements of activation of functional proteins showed increase in activation above natural state. In this example, the time periods cited were sufficient, albeit longer time periods at 75 degrees showed no negative effects; up to an hour at 50-60 degrees celsius was also useful.

Activation of certain functional proteins by the aforementioned methods was confirmed by ELISA assay (R&D Systems, Minneapolis, Minn.). The level of activated IGF-1 was measured for the plasma source employed in the above-described pig trials and also measured in the preparation of activated functional proteins obtained as described above. In each situation, the activated IGF1 in the sample was first measured prior to subjecting the sample to the assay protocol (X) and then measured after being subjected to the assay protocol (Y). The measurements are expressed as X/Y. Specifically, when the plasma source was Innomax Plasma, from Land O'Lakes the results were as follows:
Innomax IGF-1 measurements, three results were:
35/581, 25/686, 37/573 activated/total in ng/g.
Measurements of prepared activated IGF-1 were:
150/695, 119/396, 244/364 activated/total in ng/g.

It is important to understand that the assay was used in a fairly unique manner. As noted above, the first measurement was made without subjecting the sample to a an activation process. The pH change required by the ELISA protocol is used to activate the functional proteins so, theoretically, if the pH treatment step required by the ELISA protocol is skipped, the resultant measurement should be indicative of the level of activation of the growth factors in the sample. The second measurement was made after the sample was subjected to the extreme pH which is part of the ELISA protocol. Theoretically, this extreme pH should result in activation of all of the growth factors.

Thus, the present invention has been described in an illustrative manner. It is to be understood that the terminology that has been used is intended to be in the nature of words of description rather than of limitation.

Many modifications and variations of the present invention are possible in light of the above teachings. Therefore, within the scope of the appended claims, the present invention may be practiced otherwise than as specifically described.

1. A method of using at least one activated functional proteins selected from the group consisting of lysozymes, lactoferrin, growth factors, transfer factors, cytokines, and immunoglobulins to improve at least one animal health metric selected from a group consisting of weight gain, feed intake, feed efficiency, reduce medical intervention rates, and lower mortality said method comprising the step of administering an effective amount of said at least one activated functional protein wherein the level of activation of said activated functional protein is above the level found in a biological source of said at least one functional protein.

2. A method of using at least one activated functional protein from the group consisting of lysozymes, lactoferrin, growth factors, transfer factors, cytokines, and immunoglobulins to produce an improved animal supplement comprising mixing with a conventional supplement an effective amount of the at least one activated functional protein wherein the level of activation of said at least one activated functional protein is above the level found in a biological source of said at least one activated functional protein and said effective amount comprises an amount that improves one of the following metrics: feed to gain ratio, reduction of mortality, reduction in antibiotic treatments, reduction in electrolyte treatments, and improvement in average daily gain.

3. The method of claim 1 wherein the level of activation of said at least one functional protein is increased above the level of activation of said at least one functional protein found in said biological source by a method selected from a group consisting of pH adjustment, temperature adjustment, alcohol extraction, enzyme addition, ionic change, chemical addition, and pressure changes.

4. The method of claim 1 wherein the level of activation of the at least one functional protein is increased above the level of activation of said at least one functional protein found in said biological source by heat shock.

5. The method of claim 1 wherein said at least one activated functional protein are fed to young animals.

6. A method of producing an improved animal feed for supplementing the diet of an animal comprising the steps of mixing a conventional feed with a material containing at least one activated growth factor derived from a biological source wherein the level of activation of said at least one activated growth factor is higher than the level of activation of said growth factor in the biological source.

7. A composition for reducing the mortality rate of young animals comprising effective amounts of a material comprising a level of activation of at least one activated functional protein selected from the group consisting of lysozymes, lactoferrin, growth factors, transfer factors, cytokines, and immunoglobulins higher than a biological source of the at least one activated functional protein.

8. The method of claim 5 wherein said at least one activated functional protein comprises at least one growth factor and said young animal comprises a neonatal calf.

9. The method of claim 5 wherein said at least one activated functional protein comprises at least one activated growth factor and said young animal comprises a calf within three days of birth.

10. The method of claim 1 for improving growth of young pigs comprising improving a feed to gain ratio by providing a feed composition comprising said effective amount of said at least one activated functional protein.

11. The method of claim 1 for improving growth of young pigs comprising increasing an average daily weight gain by providing a feed composition comprising an said effective amount of said at least one activated functional protein.

12. The method of claim 1 wherein the step of administering comprises using said effective amount of at least one activated functional protein as a feed supplement wherein said at least one activated functional protein comprises activated TGFbeta.

13. The method of claim 1 wherein the step of administering comprises dosing the effective amount of at least one activated functional protein as an oral medicinal application.

14. The method of claim 1 wherein the step of administering comprises applying the effective amount of at least one activated functional protein as a topical application.

15. The method claimed in claim 1 wherein the step of administering comprises applying the effective amount of at least one activated functional protein as an injected medicinal application.

16. The method claimed in claim 1 wherein said animal is selected from the group consisting of: bovine, porcine, equine, canine, feline, poultry, human, and aquacultured animals.

17. The method claimed in claim 1 wherein reducing said medical intervention rates comprises reduction of the rate of at least one selected from a group consisting of antibiotic treatments and electrolyte treatments.

18. The method of claim 1 wherein said at least one functional protein is obtained from a group of sources consisting of milk, whey, colostrum, whey protein, embryonic tissue, mammal tissue, animal tissue, plasma, blood, whole blood and red blood cells, said source comprising said at least one functional protein activated to a first level and said method further comprises the step of increasing the level of activation of said at least one functional protein to a second level.
